(19) United States
(12) Patent Application Publication
(10) Pub. No.: US 2023/0218675 A1
(43) Pub. Date: Jul. 13, 2023

(54) EXOSOMES ISOLATED FROM DERMAL PAPILLA PROGENITOR CELLS, AND USE THEREOF

(71) Applicant: CELLPARK GLOBAL, Seoul (KR)

(72) Inventors: Sung Ha KIM, Seoul (KR); Hye-In CHOI, Seoul (KR)

(21) Appl. No.: 18/000,642

(22) PCT Filed: Jun. 5, 2020
(86) PCT No.: PCT/KR2020/007341

§ 371 Date: Dec. 2, 2022

(51) Int. Cl. A61K 35/36; C12N 5/071; A61P 17/14

(52) U.S. Cl. CPC A61K 35/36; C12N 5/0625; A61P 17/14; C12N 2500/02; C12N 2513/00

(57) ABSTRACT

The present invention relates to exosomes isolated from dermal papilla progenitor cells, specifically, the exosomes isolated from the dermal papilla progenitor cells which are excellent in prevention, improvement and treatment of hair loss (alopecia) and are also excellent in terms of skin improvement and wound healing effects, as well as various uses thereof.

### TECHNICAL FIELD

The present invention relates to exosomes and uses thereof, and more particularly, to exosomes isolated from dermal papilla progenitor cells and various uses thereof.

### BACKGROUND ART

Hair loss is a phenomenon in which hair falls out of the scalp through anagen, catagen and telogen in the hair cycle, and generally, hair growth and hair loss occur periodically for about 3 to 5 years. However, if the growth/hair loss balance is disrupted by different factors and the number of hairs lost per day becomes about 100 or more, it is judged as a disease called “hair loss (alopecia)”. There are factors of such alopecia known in the art, including internal factors such as heredity or male hormone degeneration and external factors caused by stress in daily life and harmful substances (lipid peroxide, etc.) accumulated on the scalp. Mostly, it is denaturation of male hormone (modification from testosterone to dihydrotestosterone).

Common methods for treating alopecia include drug treatments such as Propecia and Avodart, which are known as representative hair loss treatment drugs, and a hair transplantation method in which another person's hair is transplanted. Propecia and Avodart, which are widely used for drug treatment, were originally known as drugs for the treatment of benign prostatic hyperplasia and further proved to exhibit hair growth effects, thereby being commercially available with excellent medical efficacy. However, side effects such as erectile dysfunction for male, infertility for female, etc., have also been reported. On the other hand, the hair transplantation method is a method of transplanting one's own or another person's hair and exhibits the best effect. However, this method is temporary because it does not overcome or treat the basic cause, and entails a problem of causing pain during transplantation. In order to solve the above problems, a bio-derived material or component may be used as an active ingredient and exosomes are an example of the bio-derived material. Exosome is a vesicle composed of a lipid-bilayer and is a constituent of a substance secreted by a cell to an outside of the cell. The exosomes are known to play a role of transporting (delivering) proteins, bioactive lipids and RNA (miRNA) as biomolecules in cells, so as to implement a functional role that mediates cell-cell communication and cellular immunity. Such exosomes are also studied as a biomarker for neurological diseases such as Alzheimer's disease, and has high selective permeability to penetrate a blood-brain barrier (BBB) that separates cerebrospinal fluid and blood from each other, thereby being applied to the development of drug delivery systems such as a nanocarrier of specific drugs.

As the prior art related to the treatment of hair loss, a composition for preventing hair loss or promoting hair growth (Korean Patent Registration Publication No. 10-2112736), a composition for preventing hair loss or promoting hair growth, including irone as an active ingredient (Korean Patent Laid-Open Publication No. 10-2019-0046697) or the like. However, these are related to chemical drug therapeutics and thus have different technical characteristics from the present invention.

Meanwhile, in the case of chemical component-based therapeutics, side effects are often accompanied as well. Therefore, there is an increasing need to develop drugs that have excellent therapeutic effects for specific diseases and little side effects. Accordingly, the present inventors have studied the efficacy of exosomes and, as a result, isolated the exosomes from dermal papilla progenitor cells and attempted to complete the invention for various uses thereof.

### REFERENCES

### Patent Document

(Patent Document 1) Korean Patent Registration Publication No. 10-2112736
(Patent Document 2) Korean Patent Laid-Open Publication No. 10-2019-0046697

### Non-Patent Document

(Non-patent Document 1) Gnedeva et al., PLoS One 10, e0116892, 2015

### SUMMARY OF INVENTION

### Problem to be Solved by Invention

In order to solve the above problems, the present invention is intended to provide exosomes derived from dermal papilla progenitor cells with few side effects and excellent effects for preventing, improving or treating hair loss, skin improvement and wound healing effects, as well as a composition for prevention, improvement or treatment of hair loss, a composition for skin improvement, and a composition for treatment of wounds or scars, each of which includes the above-described exosomes as an active ingredient.

In addition, the present invention is intended to provide a filler composition including the above-described exosomes as an active ingredient.

Further, the present invention is intended to provide a method for prevention, improvement or treatment of hair loss, a method for skin improvement, and a method for treatment of wounds or scars, each of which uses the exosome-containing composition described above.

### Means for Solving Problems

In order to achieve the above objects, the present invention provides exosomes isolated from dermal papilla progenitor cells or from a culture product of dermal papilla progenitor cells.

The exosomes of the present invention are derived from dermal papilla progenitor cells (DPPCs), and the dermal papilla progenitor cells may be derived from human-derived dermal papilla progenitor cells, mouse-derived dermal papilla progenitor cells, and a culture product thereof, but it is not limited thereto.

In addition, the above-described dermal papilla progenitor cells may include dermal papilla progenitor cells differentiated or produced from human- or mouse-derived stem cells, including embryonic stem cells, adult stem cells, induced pluripotent stem cells, hematopoietic stem cells, neural stem cells, mesenchymal stem cells, and the like, or may be isolated from culture products thereof.

The culture product may refer to a culture solution obtained by culturing the human- or mouse-derived stem cells or dermal papilla progenitor cells differentiated or produced from the same in a culture medium, or a product of drying, filtering and/or concentrating the culture solution.

Further, the culture solution may be cultured for 1 to 7 days. Preferably, after culturing for 12 to 120 hours, further preferably 24 to 96 hours, more preferably 48 to 96 hours or 60 to 84 hours, and most preferably for 72 hours, the culture solution may be collected and centrifuged thus to obtain a supernatant, which is used as the culture solution, but it is not limited thereto.

In the supernatant, size exclusion chromatography, ion exchange chromatography, density gradient centrifugation, differential centrifugation, ultrafiltration, exosome precipitation, total exosome isolation kit, immune-absorbent capture, affinity method, e.g., affinity capture, affinity purification, immunoassay, microfluidic separation, or a combination thereof may be performed to extract exosomes. In addition to the above-described method, the exosomes may be separated by any method commonly used in the art for isolating exosomes.

More preferably, the culture solution may be centrifuged at 200-400×g for 5 to 20 minutes to remove the remaining cells and cell residues, followed by taking the supernatant and high-speed centrifugation thereof at 9,000-12,000×g for 60 to 80 minutes, then, collecting the supernatant again, centrifuging the same at 90,000-120,000×g for 80 to 100 minutes, and then, removing the supernatant, so as to obtain the exosomes remaining in a lower layer.

According to a specific embodiment of the present invention, the culture solution may be collected and centrifuged at 300×g for 10 minutes to remove the remaining cells and cell residues, followed by taking the supernatant, filtering the same using a 0.22 μm filter, and then, centrifuging the product by means of a high-speed centrifuge at 10,000×g and 4° C. for 70 minutes. Following this, the centrifuged supernatant may be collected again and centrifuged at 100,000×g, 4° C. for 90 minutes using an ultracentrifuge in order to remove the supernatant, thereby separating the exosomes remaining in the lower layer. The exosomes may be extracted by any method commonly used for isolating exosomes in the art other than the above-described method.

Each exosome isolated from the dermal papilla progenitor cells of the present invention may have a size of 30 to 200 nm, preferably 30 to 180 nm, and more preferably 50 to 150 nm, but it is not limited thereto.

In addition, the exosomes of the present invention may be included in an amount of 1×10¹⁶/cell to 1×10¹⁷/cell, preferably 2×10¹⁶/cell to 5×10¹⁶/cell per cell, but they are not limited thereto.

Further, the exosomes of the present invention may express any one or more markers selected from CD63, CD9 and CD81.

The expression rate of the marker is not limited, but preferably, the expression rate of CD63 among the markers may be 80% or more, and more preferably 90% or more.

Further, with regard to the exosomes of the present invention, an amount of isolated exosomes may vary depending on the culture method of dermal papilla progenitor cells. As compared to the two-dimensional culture, which is a general cell culture method, the number of isolated exosomes may vary depending on the three-dimensional culture method, hypoxic culture, or mixed culture along with other cells.

Specifically, the three-dimensional culture method refers to a method in which cells are cultured in a suspended state in a liquid medium without adhering to a culture dish. Preferably, as the dermal papilla progenitor cells, cells cultured in 10% fetal bovine serum (FBS) DMEM/F12+Glutamax (1:1) which includes 10 μg/ml FGF2, 100 units/ml penicillin and 100 μg/ml streptomycin, were used. For dermal papilla progenitor cells, after recovering 2 to 3 sub-cultured (passage) cells by trypsin, 10⁴ cells per well placed in an ultra-low cluster, 96-well culture dish, were cultured in 10% DMEM/F12+Glutamax (1:1) medium in a cell incubator for 24 hours. After culture, the used medium may be replaced with DMEM/F12+Glutamax (1:1) serum-free medium, followed by further culturing under desired culture conditions for 48 hours.

In the case of hypoxic culture, an amount of oxygen in the atmosphere may be 0.1 to 10%, and more preferably, the culturing may be conducted under 1 to 2% oxygen condition.

The mixed culture with other cells may be conducted using the same culture dish or under the same cell culture insert (Transwell, etc.) condition, preferably, the dermal papilla progenitor cells may be placed on Ultra-low cluster, 96-well plate, with an amount of 10⁴ cells per well, and cultured in a cell incubator with DMEM/F12+Glutamax (1:1) medium for 24 hours.

After the culture, the culture medium is removed, and 10⁴ keratinocytes per well may be put and cultured in the cell incubator. After 24 hours, the cells may be washed using Epilife medium without any supplement and cultured by adding the medium, wherein the incubation time may be 24 to 72 hours, and preferably 48 hours, but it is not limited thereto.

The mixed-cultured cells are skin and hair-related cells, and may include dermal papilla progenitor cells, dermal papilla cells, keratinocytes, outer root-sheath cells, melanocytes, fibroblasts, and the like, but they are not limited thereto.

The present invention may provide a composition including the exosomes as an active ingredient, and the composition may be a composition selected from pharmaceutical compositions, cosmetic compositions and food compositions.

The pharmaceutical composition in the present invention may be a formulation selected from the group including tablets, capsules, injections, creams, gels, patches, sprays, ointments, plasters, lotions, liniment agents, paste agents and Cataplasmas, etc., but it is not limited thereto.

The pharmaceutical composition of the present invention may further include a pharmaceutically acceptable carrier, lubricant, wetting agent, sweetening agent, flavoring agent, emulsifying agent, suspending agent, preservative, and the like, which are commonly used in formulation.

The pharmaceutical composition of the present invention may be administered orally or parenterally. In the case of parenteral administration, the composition may be administered through intravenous injection, subcutaneous injection, intramuscular injection, intraperitoneal injection, transdermal administration, mucosal administration and eye drop administration, topical administration, etc., but it is not limited thereto.

A suitable dosage of the pharmaceutical composition of the present invention may be differently prescribed depending on factors such as a formulation method, administration method, age, weight, sex and pathological condition of a patient, food intake, administration time, administration route, excretion rate and reaction sensitivity. Preferably, the dosage of the pharmaceutical composition of the present invention may range from 0.0001 to 100 mg/kg (body weight) based on an adult, but it is not limited thereto.

The cosmetic composition in the present invention may be produced in the form of beauty wash, lotion, essence, cream, etc., in addition, may also be produced in the form of massage cream, body lotion, body milk, bath oil, shower gel, shower cream, sunscreen, hand lotion, hair lotion, soap, shampoo, foam soap, cleansing foam, cleansing oil, cleansing cream, scalp cleaner, etc., but it is not limited thereto.

In the preparation of the cosmetic composition of the present invention, the composition may further include conventional adjuvants such as antioxidants, stabilizers, solubilizing agents, vitamins, pigments and fragrances commonly used in the production of cosmetics, as well as carriers, and may be produced in the formulation commonly used in the art for preparing cosmetics.

The food composition in the present invention may be prepared in the form of powder, granules, tablets, capsules or beverages, but it is not limited thereto. In addition, the composition may further include additives, fragrances, thickeners, stabilizers, pH adjusters, etc., which are commonly used in the art for preparing the food composition. In addition, the preparation method of the food composition may be any method commonly used for preparing food in the art, and it is not limited to specific methods.

The present invention provides a pharmaceutical composition for prevention, improvement or treatment of hair loss, a cosmetic composition for prevention or improvement of hair loss, and/or a food composition for prevention or improvement of hair loss, each of which includes the above-described exosomes as an active ingredient.

The hair loss may include androgenetic alopecia, telogen alopecia, chemical hair loss, mechanical hair loss, traumatic alopecia, pressure hair loss, genital alopecia, alopecia areata, syphilitic alopecia, seborrheic alopecia, symptomatic alopecia, scarring alopecia, congenital alopecia, circular alopecia, ringworm of the head, alopecia totalis, hypotrichosis, hereditary hypotrichosis simplex and generalized alopecia, but it is not limited thereto.

The cosmetic composition for prevention and improvement of hair loss may be any preparation selected from the group including hair tonic, hair conditioner, hair essence, hair lotion, hair nutrition lotion, hair shampoo, hair conditioner, hair treatment, hair cream, hair nutrition cream, hair moisture cream, hair massage cream, hair wax, hair aerosol, hair pack, hair nutrition pack, hair soap, hair cleansing foam, hair oil, hair drying agent, hair preservative, hair dye, hair wave agent, hair bleach, hair gel, hair glaze, hair dressing agent, hair lacquer, hair moisturizer, hair mousse and hair spray, but it is not limited thereto.

Further, the present invention provides a composition for improvement or treatment of wounds or scars, which includes the above-described exosomes as an active ingredient.

The wound refers to a wound on the skin, and specifically, may refer to damaged skin such as a diabetic foot, a pressure sore, a burn, a laceration (swept wound), or a chronic wound in which the self-healing ability is significantly deteriorated, but it is not limited thereto.

The pharmaceutical composition for treatment of wounds or scars may promote cell regeneration ability of the damaged skin or scar region and restore the same as normal skin so that the damaged skin or scars can be treated.

The composition for improvement or treatment of wounds or scars may include any one selected from a pharmaceutical composition, a cosmetic composition and a food composition.

Further, the present invention provides a pharmaceutical composition, a cosmetic composition or a food composition for skin improvement, which includes the above-described exosomes as an active ingredient.

The skin improvement may include any one or more selected from skin whitening, wrinkle improvement, elasticity enhancement, skin regeneration, skin moisturizing, anti-aging, alleviation of skin irritation, prevention or improvement of acne, prevention or improvement of atopic dermatitis, but it is not limited thereto.

Further, the present invention provides a filler composition, including the above-described exosomes as an active ingredient.

The filler composition may be used for promoting hair growth, preventing, improving or treating hair loss, improving skin, treating wounds, etc., but it is not limited thereto.

An administration method of the filler composition is not limited in a particular manner, and may include various administration methods known in the art, but injection administration is preferably used.

However, the administration method is not limited to the above-described method, and therefore, does not exclude various administration methods known in the art.

The filler composition may be injected into the skin region including the scalp, and preferably administered to the dermis. The administration may include administering the filler to the patient at a depth of about 1 mm or less from the surface of the skin. The composition may be injected at a depth of preferably, without limitation thereof, about 0.8 mm or less, and more preferably about 0.6 mm or less, or about 0.4 mm or less. The filler composition may further include, in addition to the exosomes of the present invention as an active ingredient, additives, fillers, anti-aggregative agents, lubricants, wetting agents, fragrances, emulsifiers, preservatives, etc., which are commonly used in the art for preparing fillers.

A method for preparing the filler composition may include any method for preparation of filler composition commonly used in the art, and it is not limited to any specific method.

A preparation form of the filler composition may also include any form that can be conventionally prepared for the filler composition, and it is not limited to any specific dosage form.

When the exosomes are included as an active ingredient of a pharmaceutical composition, a cosmetic composition, a food composition or a filler composition, the exosomes may be included, without limitation thereof, in the number of 1.0×10⁶ particles to 1.0×10¹² particles. When a content of the exosomes, that is, the number of exosomes is less than 1.0×10⁶ particles, effects thereof may be insignificant. When the content exceeds 1.0×10¹² particles, it may cause toxicity to the human body.

Further, the present invention may provide a method for prevention, improvement or treatment of hair loss, a method for skin improvement, a method for treatment of wounds or scar, and the like, by application of the exosomes.

### Advantageous Effects

The present invention relates to exosomes isolated from dermal papilla progenitor cells, and may provide the exosomes isolated from dermal papilla progenitor cells, which are excellent in preventing, improving and treating hair loss and also exhibit excellent skin improvement and wound healing effects, as well as various uses thereof.

### BRIEF DESCRIPTION OF DRAWINGS

FIG. 1 shows extraction values of exosomes from cells according to the present invention.

FIG. 2 shows marker expression distributions of the exosomes according to the present invention for each derived cell.

FIG. 3 shows evaluation result of cell proliferation of the exosomes according to the present invention.

FIGS. 4A and 4B show evaluation result of effects (hair follicle length growth) of the exosomes according to the present invention on the hair follicles.

FIG. 5 shows evaluation result of effects of the exosomes according to the present invention on the hair growth cycle.

FIG. 6 shows evaluation result of cell proliferation of the exosomes according to the present invention in the hair follicles.

FIG. 7 shows evaluation result of growth factor expression of the exosomes according to the present invention in the dermal papilla cells.

FIGS. 8A, 8B and 8C show evaluation result of hair growth-related gene expression of the exosomes according to the present invention.

FIGS. 9A and 9B show evaluation result of aging recovery of the exosomes according to the present invention in aged dermal papilla cells.

FIG. 10 shows evaluation result of cell mobility of the exosomes according to the present invention.

### MODE FOR CARRYING OUT INVENTION

Hereinafter, the present invention will be described in detail by way of examples and experimental examples.

However, the following examples and experimental examples are merely illustrative of the present invention, and the content of the present invention is not limited to the following examples and experimental examples.

### Examples 1 and 2

In order to isolate the exosomes of the present invention, dermal papilla progenitor cells were prepared with reference to the following document [Gnedeva et al., PLoS One 10, e0116892, 2015]. Using the dermal papilla progenitor cells prepared according to the above-described method, the cells were cultured in a cell incubator under a hypoxic condition of 1% oxygen in Example 1 and under a normal culture condition of 5% carbon dioxide, 37° C. in Example 2, respectively.

The dermal papilla progenitor cells were cultured on an ultra-low cluster, 96-well culture dish at 10⁴ cells per well in 10% DMEM/F12+Glutamax (1:1) medium for 24 hours. After the culture, the above medium was replaced with DMEM/F12+Glutamax (1:1) serum-free medium, followed by culturing for 48 hours. Thereafter, the medium was collected and centrifuged at 300×g for 10 minutes to remove remaining cells and cell residues, while the supernatant was taken, filtered using a 0.22 μm filter, and then centrifuged at 10,000×g and 4° C. for 70 minutes using a high speed centrifuge. The centrifuged supernatant was collected again and centrifuged at 100,000×g and 4° C. for 90 minutes using an ultracentrifuge in order to remove the supernatant, thereby isolating and obtaining the exosomes remaining in the lower layer.

### Comparative Examples 1 and 2

The exosome isolation method was the same as in Examples 1 and 2, except that the exosomes were isolated and obtained using fibroblasts in Comparative Example 1 or dermal papilla cells in Comparative Example 2 instead of the dermal papilla progenitor cells.

### <Experimental Example 1> Evaluation of Characteristics (Exosome Size) of the Exosomes According to the Present Invention

In order to evaluate the characteristics of the exosomes of the present invention, the size of the exosomes isolated from dermal papilla progenitor cells was analyzed using the examples 1 and 2. As a result, the exosomes of the present invention were distributed between 50 and 150 nm, and the average size was found to be 63.6 nm.

### <Experimental Example 2> Evaluation of Characteristics (Number of Exosomes Extracted Per Cell) of the Exosomes According to the Present Invention

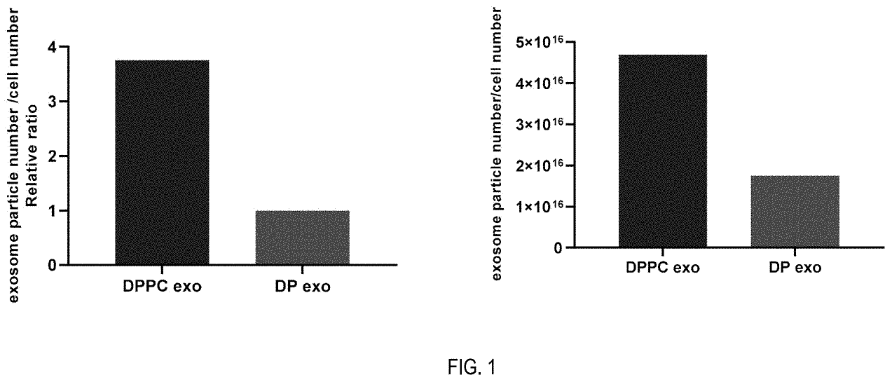

In order to evaluate the characteristics of the exosomes of the present invention, the number of exosomes secreted per cell was comparatively evaluated using the examples and Comparative Example 2. As a result, the exosomes secreted from the dermal papilla progenitor cells according to the present invention were 5×10¹⁶/cell, and the exosomes secreted from the dermal papilla cells of Comparative Example 2 were 2×10¹⁶/cell, such that the number of exosomes secreted from the dermal papilla progenitor cells according to the present invention was found to be high 2.5 times or more (FIG. 1).

### <Experimental Example 3> Evaluation of Characteristics of the Exosomes According to the Present Invention (Confirmation of Expression Markers

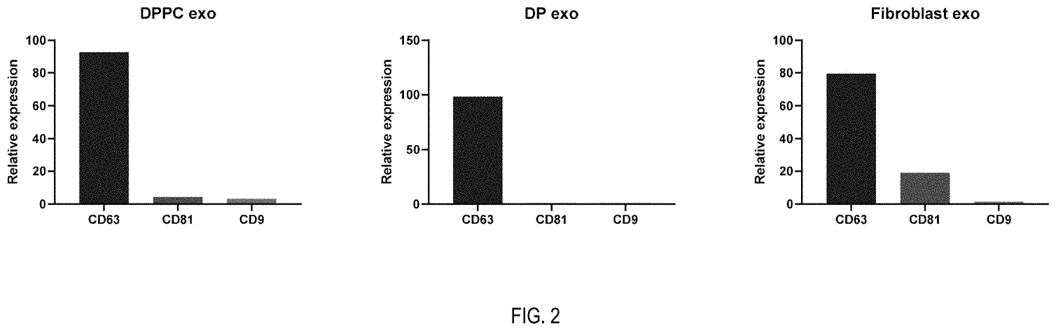

In order to evaluate the characteristics of the exosomes of the present invention, a distribution of markers expressed in the exosomes was evaluated using the examples and Comparative Examples 1 and 2. As a result of the evaluation, exosome expression markers CD63, CD9 and CD81 were commonly expressed in each cell, but expression rates for each marker were different. With regard to the exosomes of dermal papilla progenitor cells, which is the example of the present invention, the expression rate of CD63 was about 90-95%. On the other hand, with regard to the exosomes of the fibroblasts of Comparative Example 1, the expression rates of CD63, CD81 and CD9 were about 80%, 19% and 1%, respectively. With regard to the exosomes of the dermal papilla cells of Comparative Example 2, CD63 was found to be 98% or more, and the expression rates of CD9 and CD81 were found to be very low (FIG. 2).

### <Experimental Example 4> Evaluation of Cell Proliferation

In order to evaluate hair growth and skin improvement effects of the exosomes according to the present invention, dermal papilla cells (DPCs) which are known to be important for hair follicle formation and hair growth, outer root-sheath cells (ORSCs), and keratinocytes (KCs) as skin cells were subjected evaluation of cell proliferation.

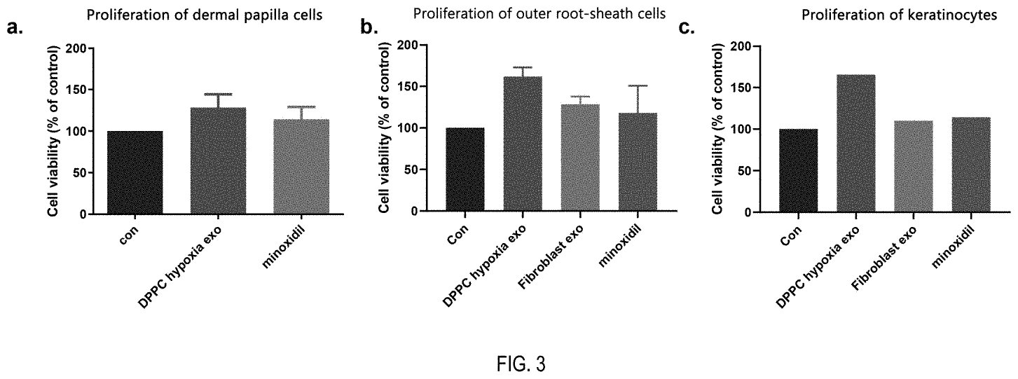

The control did not have any treatment, the positive control was treated with minoxidil used for hair loss treatment, and the comparative example was treated with fibroblast-derived exosomes. Evaluation results thereof are shown in FIG. 3. An experimental method used herein was conducted as follows: 5×10³ dermal papilla cells per well were cultured on a 96-well culture dish (well plate) in 10% DMEM medium for 24 hours; after 24 hours, the medium was replaced with a serum-free DMEM medium while the cells were treated with the exosomes (1.0×10⁹ particles) of Example 1 and minoxidil (1 μM) as a positive control; and, after 48 hours of the culture, 75 μl of serum-free medium and 25 μl of MTT solution were added, followed by further culturing for 3 hours and then measuring absorbance at 570 nm in order to confirm cell proliferation. A cell proliferation rate refers to an increase rate of cell proliferation indicated on a graph by comparing measurement results of the absorbance to those of the negative control.

The outer root-sheath cells and keratinocytes of the hair follicles were cultured in Epilife medium at 1×10⁴ cells per well in a 96 well plate for 24 hours. After culturing for 24 hours, the medium was replaced with a medium without supplements while the cells were treated with the exosomes (1.0×10⁹ particles) of Example 1 and minoxidil (1 μM) as a positive control, followed by the above evaluation process under the same conditions as above.

As a result of the evaluation, with regard to Example 1 according to the present invention, proliferation was active in all dermal papilla cells, outer root-sheath cells and keratinocytes, in particular, it could be seen that Example 1 has the excellent cell proliferation rate compared to minoxidil used as a hair loss therapeutic agent (FIG. 3).

### <Experimental Example 5> Evaluation of Hair Follicle Growth Effects Through Human Hair Follicle Organ Culture Test

In order to evaluate effects of the exosomes of the present invention on human hair follicle growth, a human hair follicle organ culture test (ex vivo hair follicle organ culture) was conducted. More specifically, scalp tissues were isolated into the unit of hair follicles, and the remaining hair follicles were used after cutting the part below the sebaceous gland of the isolated hair follicles. The prepared hair follicles were treated with Examples 1 and 2, and minoxidil as a positive control, respectively, and lengths of the hair growing on the 3rd and 6th days were measured, followed by comparing the measured results to the negative control in order to evaluate a length growth and a growth cycle of human hair follicles.

### 5-1. Hair Follicle Length Growth Evaluation 1

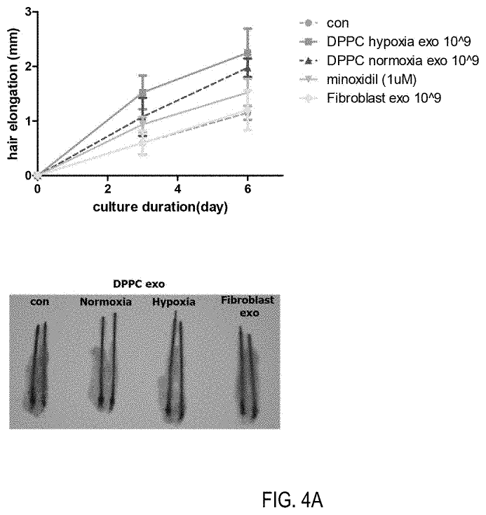

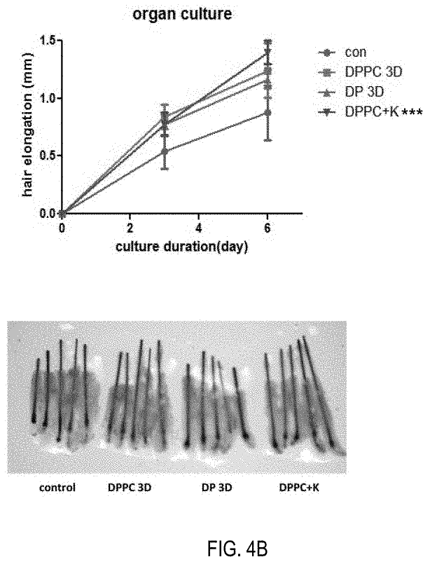

In order to evaluate effects of the dermal papilla progenitor cell-derived exosomes isolated with different cells and culture conditions on the length growth of hair follicles, human hair follicle tissues were cultured in Williams E culture medium containing 2 mM L-glutamine, 100 U/ml streptomycin, 10 ng/ml hydrocortisone and 10 μg/ml insulin, the exosomes (1.0×10⁹ particles) of Example 1, and the exosomes (1.0×10⁹ particles) of Example 2, minoxidil (1 μM) as a positive control and the exosomes (1.0×10⁹ particles) of Comparative Example 2, respectively, were used for treatment of the cells, followed by culturing the cells in a carbon dioxide incubator at 37° C. The lengths of the hair follicles grown on the 3rd and 6th days of the culture, respectively, were measured using a microscope ruler. Evaluation results thereof are shown in FIG. 4A.

As a result of the evaluation, the control and Comparative Example 1 without any treatment showed the growth of hair by 1 mm for 6 days, while the positive control showed the growth of hair by about 1.5 mm. On the other hand, Example 1 with hypoxic culture showed the growth of hair by about 2.2 mm for 6 days, while Example 2 with general culture showed the growth of hair by 1.8 mm. From the above results, it can be seen that the exosomes isolated after hypoxic culture is more excellent in hair growth effects by 20% or more as compared to the positive control.

### 5-2. Hair Follicle Length Growth Evaluation 2

In order to evaluate effects of the dermal papilla progenitor cell-derived exosomes isolated with different cells and culture methods on the length growth of hair follicles, human hair follicle tissues were cultured in Williams E culture medium containing 2 mM L-glutamine, 100 U/ml streptomycin, 10 ng/ml hydrocortisone and 10 μg/ml insulin, the exosomes (1.0×10⁹ particles) of Example 2 with three-dimensional culture, the exosomes (1.0×10⁹ particles) of Comparative Example 2 with three-dimensional culture, and the exosomes (1.0×10⁹ particles) of Example 2 with three-dimensional culture and mixed culture along with the keratinocytes, respectively, were used for treatment of the cells, followed by culturing the cells in a carbon dioxide incubator at 37° C. The lengths of the hair follicles grown on the 3rd and 6th days of the culture, respectively, were measured using a microscope ruler. Evaluation results thereof are shown in FIG. 4B.

As a result of the evaluation, the exosomes (1.0×10⁹ particles) cultured by a general culture method and then isolated in Example 2 showed the growth of hair by about 0.6 mm for 6 days, while the exosomes (1.0×10⁹ particles) three-dimensionally cultured and then isolated in Comparative Example 2 showed the growth of hair by about 1.1 mm. On the other hand, the exosomes (1.0×10⁹ particles) three-dimensionally cultured and then isolated in Example 2 showed the growth of hair by 1.2 mm, while the exosomes (1.0×10⁹ particles) three-dimensionally cultured and then mixed-cultured along with the keratinocytes and then isolated in Example 2 showed the growth of hair by about 1.4 mm. Therefore, it can be confirmed that, unlike the general culture method, the exosome, which was cultured according to three-dimensional culture and then isolated, is more superior in terms of hair follicle length growth rate. In addition, it can be seen that there is a synergistic effect when treated along with keratinocytes.

### 5-3. Hair Growth Cycle Evaluation

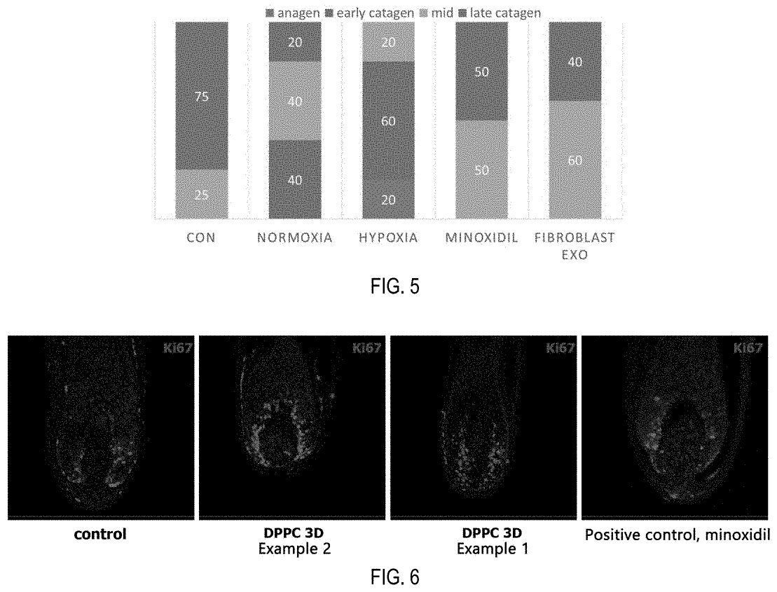

In order to evaluate effects of the present invention on the hair growth cycle, only the anagen hair follicles were isolated from the hair follicle tissue and the exosomes (1.0×10⁹ particles) isolated in each of Examples 1 and 2, and the exosomes (1.0×10⁹ particles) isolated in each of the controls, the positive control (1 μM) and the comparative example were used for treatment of the hair follicles. Then, in order to evaluate the growth cycle of hair follicles, an experiment was conducted with reference to the following document [Langan et al., Experimental dermatology, 2015]. Growth cycle classification criteria were classified by comparing a distance between the hair shaft and dermal papilla cells after observing human hair follicles under a microscope. The growth cycle was divided into anagen and catagen phases 1 to 3 by scores, and measured results are shown in FIG. 5.

As a result of the evaluation, most of the hair follicles treated with the control were converted into the catagen without the anagen, while the hair follicles treated with the exosomes isolated in Comparative Example 1 had a shorter catagen than the control but did not exhibit the anagen. The hair follicles treated with minoxidil also showed similar results to the hair follicles treated with the exosomes isolated in Comparative Example 1. In contrast, with regard to the hair follicles treated with the exosomes isolated in Example 2 (normal culture condition) of the present invention, the catagen was very short at 20%, and the initial catagen was maintained at 40% or more. The hair follicles treated with the exosomes isolated in Example 1 (hypoxic condition) did not show the catagen at all, but had 60% of initial anagen and 20% of anagen, thereby indicating that the hair continues to grow.

### 5-4. Evaluation of Hair Matrix Cell Proliferation in Hair Follicles

In order to evaluate effects of the exosomes of the present invention on the proliferation of hair matrix cells involved in direct length growth of hair, only anagen hair follicles were isolated from the hair follicle tissues and used in the present experiment. The human hair follicle tissues were cultured using Williams E culture medium containing 2 mM L-glutamin, 100 U/ml streptomycin, 10 ng/ml hydrocortisone and 10 μg/ml insulin in a carbon dioxide incubator at 37° C. for 2 days after treating the tissues with a control, a positive control (1 μM), and the exosomes (1.0×10⁹ particles) three-dimensionally cultured and then isolated in Examples 1 and 2. After culturing, the human hair follicles were frozen and cut, followed by immune-fluorescence staining for Ki-67. As the number of Ki-67 positive cells in the hair matrix cells is increased, hair follicle growth was evaluated to be increased. Evaluation results thereof are shown in FIG. 6.

As a result of the evaluation, it can be seen that more Ki-67-positive cells (red) were expressed in the hair follicle tissues treated with the exosomes isolated in Examples 1 and 2 of the present invention as compared to the control and minoxidil as the positive control.

### <Experimental Example 6> Evaluation of Hair Growth-Related Growth Factor Expression

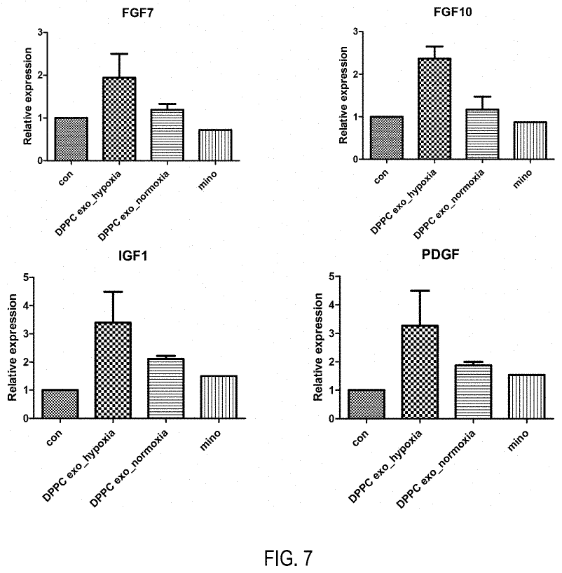

In order to evaluate effects of the exosomes of the present invention on hair growth, the expression of genes important for hair growth in dermal papilla cells was investigated. On a 6-well culture dish (well plate), 5.0×10⁴ dermal papilla cells per well were cultured in 10% DMEM culture medium for 24 hours. After washing twice using a serum-free DMEM medium, the cells were cultured in the serum-free DMEM medium for 24 hours. The exosomes (1.0×10⁹ particles) isolated in Examples 1 and 2, a control and a positive control (1 μM) were used for treatment of the cells for 2 hours, respectively, followed by extracting RNA and synthesizing cDNA. It was evaluated whether FGF7, FGF10, IGF1 and PDGF genes, which are growth factors involved in hair growth, are expressed or not through real-time PCR. Evaluation results thereof are shown in FIG. 7.

As a result of the evaluation, it can be seen that the exosomes isolated in Examples 1 and 2 of the present invention have higher expression rates of all growth factors compared to minoxidil, that is, the positive control. In particular, it could be seen that the expression of the growth factors treated with the exosomes isolated in Example 1 was the highest.

### <Experimental Example 7> Evaluation of Gene Expression Related to Hair Follicle Formation Ability

In order to evaluate how much the exosomes of the present invention maintains hair follicle formation ability, the expression of proteoglycan, a Wnt target gene and a dermal papilla cell marker gene, respectively, which are genes involved in the hair follicle formation ability of dermal papilla cells, was investigated.

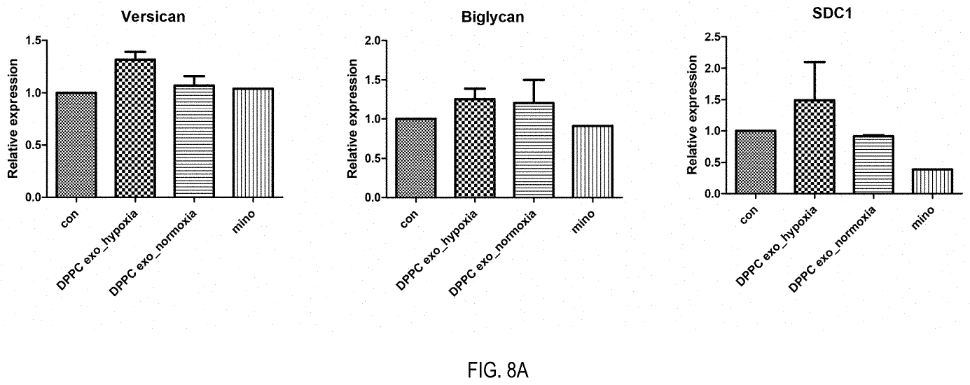

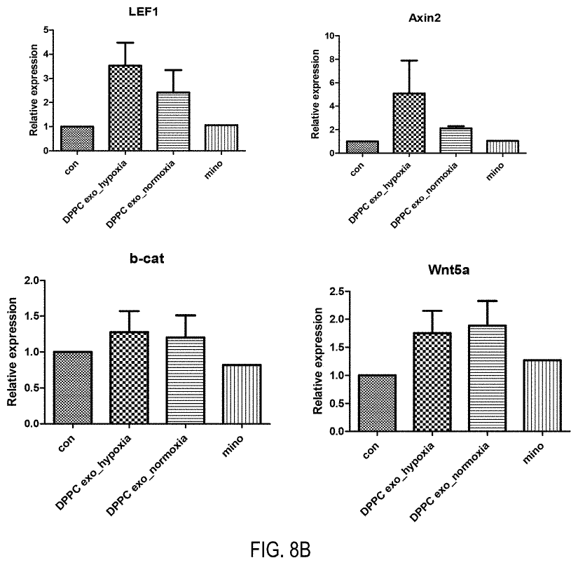

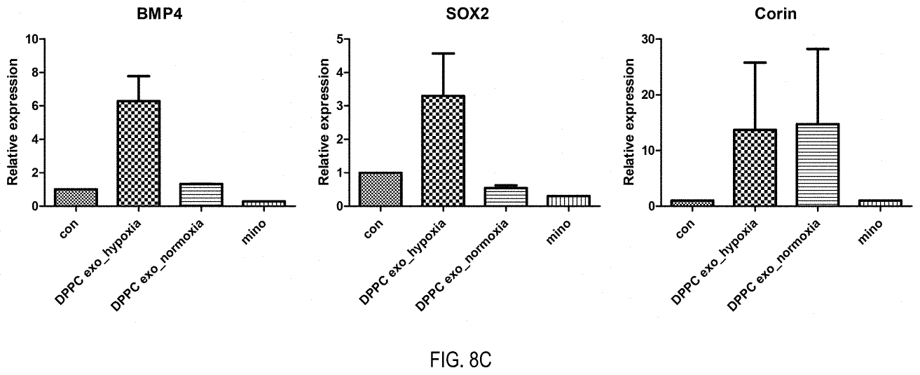

On a 6-well culture dish (well plate), 5.0×10⁴ dermal papilla cells per well were cultured in 10% DMEM culture medium for 24 hours. After washing twice using a serum-free DMEM medium, the cells were cultured in the serum-free DMEM medium for 24 hours. The exosomes (1.0×10⁹ particles) isolated in Examples 1 and 2, a control and a positive control (1 μM) were used for treatment of the cells for 4 hours, respectively, followed by extracting RNA and synthesizing cDNA. It was evaluated whether each of the genes is expressed or not through real-time PCR. Evaluation results thereof are shown in FIGS. 8A to 8C.

### 7-1. Evaluation of Proteoglycan-Related Gene Expression

Proteoglycans are polymorphic macromolecules present in skin and hair, and are known to interact with growth factors and collagen. Further, it has been found that the above macromolecules are decreased in telogen hair follicles while being increased in anagen hair follicles in the hair growth cycle (Couchman, 2017, Journal of Dermatological Science). Using the dermal papilla cells treated with the exosomes isolated in Examples 1 and 2, a control and a positive control, respectively, the expression of Versican, Biglycan and Syndecanl genes involved in proteoglycan expression was evaluated through real-time PCR. Evaluation results thereof are shown in FIG. 8A.

As a result of the evaluation, the exosomes isolated in Example 2 of the present invention demonstrated the expression of genes at a level equal to or higher than that of the positive control. In particular, it can be seen that, with regard to the exosomes isolated in Example 1 of the present invention, the expression of all genes exhibits higher than that of the positive control.

### 7-2. Evaluation of Wnt Target Gene Expression

The Wnt signaling system is a key material in the process of hair follicle formation and differentiation, and it is well known that the activation of related genes is important for entering the anagen from the telogen in the hair follicle growth cycle (Lim & Nusse, 2013). In order to confirm whether the exosomes of the present invention regulate the expression of major target genes in the Wnt signaling system, the dermal papilla cells treated with the exosomes isolated in Examples 1 and 2, the control and the positive control, respectively, were used to evaluate the expression rates of LEF1, Axin2, beta-catenin and Wnt5a gens, which are involved in the expression of Wnt target genes through real-time PCR. The evaluation is to indicate values in proportion to gene expression types of the negative control on a graph. Evaluation results thereof are shown in FIG. 8B.

As a result of the evaluation, the exosomes isolated in Examples 1 and 2 of the present invention demonstrated higher expression rates of LEF1, Axin2, beta-catenin and Wnt5a as compared to the control and the positive control. In particular, it can be seen that the expression rate of each of LEF1 and Axin2 exhibits high 2 times or more, while beta-catenin and Wnt5a exhibit high 30-40% or more, as compared to the positive control.

### 7-3. Evaluation of Dermal Papilla Cell Representative Gene Expression

It is known that, with increase in the expression rates of BMP4, SOX1 and Corin, which are well known as markers of dermal papilla progenitor cells, the hair follicles formation ability is increased. Using the dermal papilla cells treated with the exosomes isolated in Examples 1 and 2, the control and the positive control, respectively, the expression of BMP4, SOX2 and Corin genes, which are representative dermal papilla cell genes, was evaluated through real-time PCR. Results thereof are shown in FIG. 8C.

As a result of the evaluation, it could be seen that, with regard to the exosomes isolated in Examples 1 and 2 of the present invention, the expression rates of BMP4, SOX2 and Corin genes exhibited higher at least 3 times or more and at most 10 times or more than that of the control and the positive control.

### <Experimental Example 8> Evaluation of Aging Recovery in Aged Dermal Papilla Cells

As the dermal papilla cells are repeatedly sub-cultured, the cells become aged, that is, senescent (cellular senescence) and thus lose an ability of forming hair follicles. It was investigated whether the exosomes of the present invention are effective in the hair follicle formation ability and cell aging recovery of aged dermal papilla cells that have lost the hair follicle formation ability.

### 8-1. Evaluation of Hair Follicle Formation Ability (“Rejuvenation”

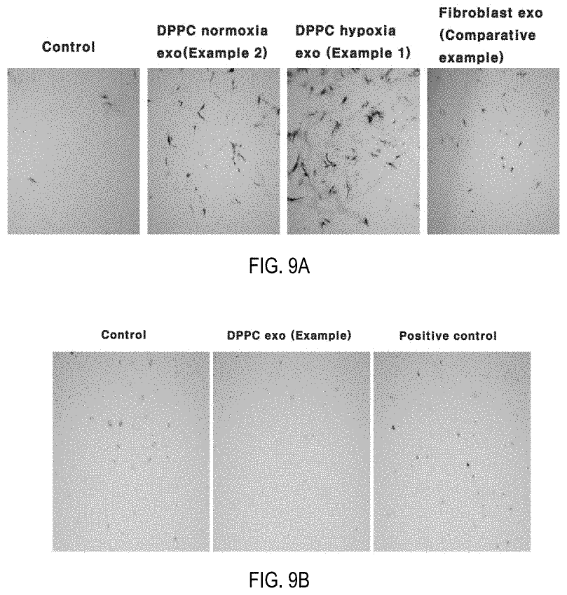

Alkaline phosphatase (AP) is a representative marker of hair follicle-formation ability and, when dermal papilla cells become aged, the activity of alkaline phosphatase is decreased. On a 6-well culture dish (well plate), 3.0×10⁴ dermal papilla cells were cultured in 10% DMEM for 24 hours. After washing twice with a serum-free DMEM medium, the exosomes (1.0×10⁹ particles) isolated in Examples 1 and 2, Comparative Example 1 and the control, respectively, were used for treatment of the cells. After 48 hours, the cells were washed twice with tris-buffered saline (TBS) and fixed with cold acetone for 10 minutes, followed by reaction for 10 minutes while adding a mixed solution of NBT/BCIP in TN (0.01M Tris-HCl and 0.1M NaCl, pH 8.0) in a ratio of 1:50 thereto. After washing twice again with tris-buffered saline (TBS), the product was subjected to observation under a microscope, and the activity of AP could be confirmed by a purple staining extent. Results thereof are shown in FIG. 9A.

As a result of the evaluation, a ratio of cells stained with purple exhibited higher in the exosomes isolated in Examples 1 and 2 of the present invention than that of the exosomes isolated in the control and Comparative Example 1. Therefore, it can be seen that the exosomes of the present invention increase the activity of AP and also increase the hair follicle formation ability.

### 8-2. Evaluation of Anti-Aging in Dermal Papilla Cells

In order to investigate whether the exosomes of the present invention are effective in cellular senescence recovery (rejuvenation) of aged dermal papilla cells, senescence-associated β-galactosidase (SA-β-gal) as a cellular senescence marker was subjected to staining. On a 6-well culture dish (well plate), 3.0×10⁴ dermal papilla cells were cultured in 10% DMEM for 24 hours. After washing twice with a serum-free DMEM medium, the exosomes (1.0×10⁹ particles) isolated in Example 1, a control and a positive control, that is, minoxidil (1 uM) were used for treatment of the cells for 48 hours. The treated cells were stained according to the protocol using a senescence β-galactosidase staining kit. It can be confirmed that the aging of the cells is increased as the SA-β-gal staining extent (blue) is increased. Results thereof are shown in FIG. 9B.

As a result of the evaluation, it was confirmed that a ratio of cells stained with SA-β-gal was low in the exosomes isolated in Example 1 of the present invention as compared to the control and the positive control. Therefore, it could be seen that the exosomes isolated in Example 1 has excellent anti-aging effects through suppression of cell aging.

### <Experimental Example 9> Evaluation of Cell Motility

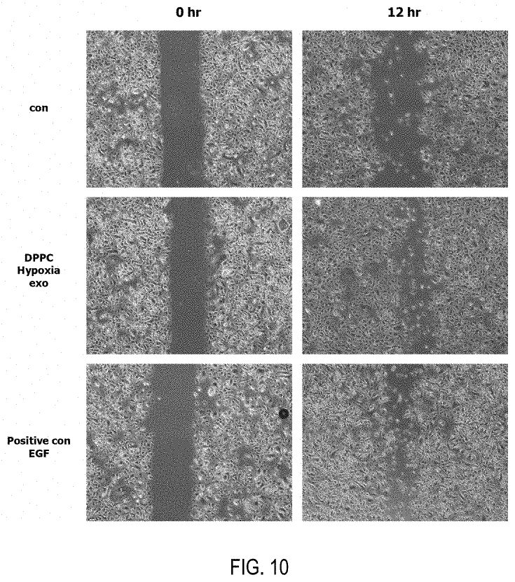

In order to evaluate effects of the exosomes according to the present invention on wound healing and skin improvement, the cell migration ability (“motility”) of keratinocytes was investigated. After placing 1.0×10⁵ keratinocytes per well on a 6-well culture dish (well plate), the cells were cultured for 24 hours. The cells in culture were scraped on the culture dish using a sterile 10 μl tip to make a wound, and then washed with phosphate buffer saline (PBS). In Epilife medium, the cells were treated with the exosomes (1.0×10⁹ particles) isolated in Example 1, a control and a positive control, that is, epidermal growth factor (EGF), which is an epidermal growth factor, respectively. After 12 hours, a degree of cell motility was observed using a microscope. The cell motility was evaluated by observing a degree of filling the wounded area under the microscope. Results thereof are shown in FIG. 10.

As a result of the evaluation, it could be seen that the cell motility was increased by the exosomes isolated in Example 1 which in turn reduced the wounded area as compared to the control. Further, it could also be seen that the cell motility was almost similar to that of the positive control, that is, EGF.

1. An exosome isolated from dermal papilla progenitor cells or from a culture product of dermal papilla progenitor cells.

2. The exosome according to claim 1, wherein the dermal papilla progenitor cells are derived from embryonic stem cells, adult stem cells, induced pluripotent stem cells, hematopoietic stem cells, neural stem cells or mesenchymal stem cells.

3. The exosome according to claim 1, wherein the dermal papilla progenitor cells are differentiated or prepared from human-derived stem cells, induced pluripotent stem cells or adipose stem cells.

4. The exosome according to claim 1, wherein the exosome has a size of 50 to 150 nm.

5. The exosome according to claim 1, wherein the exosome expresses CD63, CD9, CD81 or combinations thereof.

6. The exosome according to claim 5, wherein an expression rate of CD63 is at least 80%.

7. (canceled)

8. The exosome according to claim 1, wherein the exosome is obtained by:
a) centrifuging a culture of dermal papilla progenitor cells at 200 to 400×g for 5 to 20 minutes to obtain a supernatant;
b) removing the remaining cells and cell residues remaining in the centrifuged culture;
c) centrifuging the supernatant of a) at 9,000 to 12,000×g for 60 to 80 minutes;
d) collecting a supernatant obtained after the centrifuging of c); and
e) centrifuging the supernatant collected in d) at 90,000 to 120,000×g for 80 to 100 minutes.

9. The exosome according to claim 1, wherein the dermal papilla progenitor cells are cultured under 1 to 2% oxygen condition.

10. The exosome according to claim 1, wherein the dermal papilla progenitor cells are three-dimensionally cultured or mixed-cultured along with keratinocytes, outer root-sheath cells, melanocytes, fibroblasts or combinations thereof.

11. A pharmaceutical composition for prevention, improvement or treatment of hair loss, the composition comprising the exosome according to claim 1 as an active ingredient.

12. The pharmaceutical composition according to claim 11, wherein the composition comprises 1×10⁶ particles to 1×10¹² particles of the exosome.

13. The pharmaceutical composition according to claim 11, wherein the hair loss is androgenetic alopecia, telogen alopecia, chemical hair loss, mechanical hair loss, traumatic alopecia, pressure hair loss, genital alopecia, alopecia areata, syphilitic alopecia, seborrheic alopecia, symptomatic alopecia, scarring alopecia, congenital alopecia, circular alopecia, ringworm of the head, alopecia totalis, hypotrichosis, hereditary hypotrichosis simplex generalized alopecia or combinations thereof.

14. A method for prevention, improvement or treatment of hair loss, comprising administering the pharmaceutical composition according to claim 11 to a subject in need thereof.

15. (canceled)
